(19) United States
(12) Patent Application Publication
(10) Pub. No.: US 2026/0062411 A1
(43) Pub. Date: Mar. 5, 2026

(54) NOVEL SALT OF SUBSTITUTED 5-FLUORO-1H-PYRAZOLOPYRIDINES AND ITS USES

(71) Applicant: TORRENT PHARMACEUTICALS LIMITED, Ahmedabad (IN)

(72) Inventors: Sunil Sadanand Nadkarni, Gandhinagar (IN); Ramesh Chandra Gupta, Gandhinagar (IN); V.Srinivas Goud, Gandhinagar (IN); Niraj Krishna Naikade, Gandhinagar (IN); Ashish Upadhyaya, Gandhinagar (IN)

(21) Appl. No.: 19/103,578

(22) PCT Filed: Aug. 17, 2023
(86) PCT No.: PCT/IB2023/058243

§ 371 Date: Feb. 13, 2025

(30) Foreign Application Priority Data
Aug. 18, 2022 (IN) 202221047422

(51) Int. Cl. C07D 471/04

(52) U.S. Cl. CPC C07D 471/04

(57) ABSTRACT

The present invention provides a novel 5-fluoro-1-[(2-fluorophenyl)methyl]-1H-pyrazolo [3, 4-b]pyridine-3-carboximidamide formate compound of formula (IV) and process for preparation thereof. The present invention further provides for the use of compound of formula (IV) for the preparation of vericiguat compound of formula I. The present invention also relates to process for the preparation of vericiguat Modification form II, vericiguat Modification form III, vericiguat monohydrate and dihydrate.

### FIELD OF THE INVENTION

The present invention provides a novel 5-fluoro-1-[(2-fluorophenyl)methyl]-1H-pyrazolo[3, 4-b]pyridine-3-carboximidamide formate compound of formula (IV) and process for the preparation thereof.

The present invention further provides for the use of compound of formula (IV) for the preparation of vericiguat compound of formula I.

The present invention also relates to a process for the preparation of vericiguat Modification form II, vericiguat Modification form III, vericiguat monohydrate and dihydrate.

### BACKGROUND OF THE INVENTION

The compound of the formula (I) acts as a stimulator of soluble guanylate cyclase and can be used as an agent for prophylaxis and/or treatment of cardiovascular disorders, for example for treatment of hypertension and heart failure, including chronic heart failure, heart failure with reduced ejection fraction (HFrEF), and heart failure with preserved ejection fraction (HFpEF), stable and unstable angina pectoris, peripheral and cardiac vascular disorders, of arrythmias, for treatment of thromboembolic disorders and ischaemias such as myocardial infarction, stroke, transitory and ischaemic attacks, peripheral perfusion disorders, prevention of restenoses such as after thrombosis therapy, percutaneous transluminal angioplasty (PTA), percutaneous transluminal coronary angioplasty (PTCA), bypass, and for treatment of arteriosclerosis, asthmatic disorders and diseases of the urogenital system, for example prostate hypertrophy, erectile dysfunction, female sexual dysfunction, osteoporosis, glaucoma, pulmonary hypertension, gastroparesis, scleroderma and incontinence.

U.S. Pat. No. 8,420,656 (also referred to herein as “the '656 patent”) describes a process for preparing vericiguat (Refer Scheme 1 to 3 of “the '656 patent”). The Scheme 2 specifically discloses use of ammonium chloride and acetic acid for the preparation of 5-fluoro-1-[(2-fluorophenyl)methyl]-1H-pyrazolo[3, 4-b]pyridine-3-carboximidamide acetate. The disclosed process involves tedious workup process and affords low yield.

U.S. Pat. Nos. 8,802,847 and 10,633,356 (also referred to herein as “the '847” and “the '356”) specifically discloses use of ammonium chloride for the preparation of 5-fluoro-1-[(2-fluorophenyl)methyl]-1H-pyrazolo[3, 4-b]pyridine-3-carboximidamide hydrochloride, but also results in low yield. The '847 also discloses the compound of formula (I) may exist in various crystal forms, hydrates and solvates thereof. However, the US '847 and '356 dose not discloses the process for the preparation of Form II, III, monohydrate and dihydrate.

WO 2021254981 A1 also discloses use of ammonium chloride for the preparation of 5-fluoro-1-[(2-fluorophenyl)methyl]-1H-pyrazolo[3, 4-b]pyridine-3-carboximidamide hydrochloride, but with low yield.

The prior art processes for the preparation of vericiguat and its intermediates discloses tedious methods like column chromatography, multiple extractions, with low yield and quality.

The inventors of a present invention have developed cost effective, non and or less tedious process resulting in high yield.

The inventors of a present invention have also developed a simple, safe, efficient, economical, industrially feasible and scalable process for the preparation of vericiguat and its intermediates.

### SUMMARY OF THE INVENTION

In one aspect, the present invention provides a novel 5-fluoro-1-[(2-fluorophenyl)methyl]-1H-pyrazolo[3, 4-b]pyridine-3-carboximidamide formate compound of formula (IV).

In another aspect, the present invention provides use of compound of formula (IV) for the preparation of vericiguat compound of formula I.

In still another aspect, the present invention provides a novel process for preparing a compound of formula (IV), comprising reacting compound of formula (III) in presence of ammonium formate in an alcohol and base to form a compound of formula (IV).

In another aspect, the present invention provides a process for the preparation of vericiguat compound of formula I comprises converting a compound of formula IV to vericiguat compound of formula I.

In another aspect, the present invention provides a process for preparation of vericiguat modification form II comprising;
a) providing solution of vericiguat or solvate thereof in a solvent;
b) adding a solvent;
c) isolating vericiguat modification Form II.

In another aspect, the present invention provides a process for preparation of vericiguat modification form II comprising;
I) providing a wet vericiguat or solvate thereof;
II) isolating vericiguat modification Form II.

In another aspect, a process for preparation of wet vericiguat or solvate thereof of step (I) as used in above aspect comprises;
i) providing a solution of vericiguat or solvate thereof in a solvent;
ii) combining a solvent to form wet solid after filtration;
iii) providing a wet solid from the wet solid of step ii in a solvent.
iv) providing a wet vericiguat or solvate thereof from the wet solid of step iii in a solvent.

In another aspect, the present invention provides a process for preparation of vericiguat modification form III comprising;
a) providing a suspension of vericiguat or solvate thereof in a solvent;
b) heating the solution;
c) isolating vericiguat modification Form III.

In another aspect, the present invention provides a process for preparation of vericiguat monohydrate comprising;
a) providing a solution of vericiguat or solvate thereof in a solvent;
b) adding a mixture of solvent;
d) isolating vericiguat monohydrate.

In another aspect, the present invention provides a process for preparation of vericiguat dihydrate comprising;
a) providing a solution of vericiguat or solvate thereof in a solvent;
b) adding a solvent;
c) isolating vericiguat dihydrate.

### BRIEF DESCRIPTION OF THE FIGURES

FIG. 1: X-ray diffraction pattern (XRD) of vericiguat Modification form II.

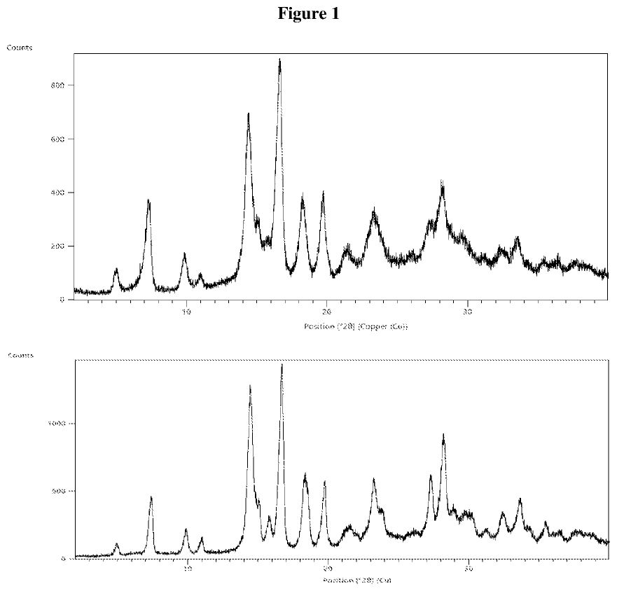

FIG. 2: X-ray diffraction pattern (XRD) of 5-fluoro-1-[(2-fluorophenyl)methyl]-1H-pyrazolo[3, 4-b]pyridine-3-carboximidamide formate compound of formula (IV).

FIG. 3: X-ray diffraction pattern (XRD) of vericiguat as per Example 21.

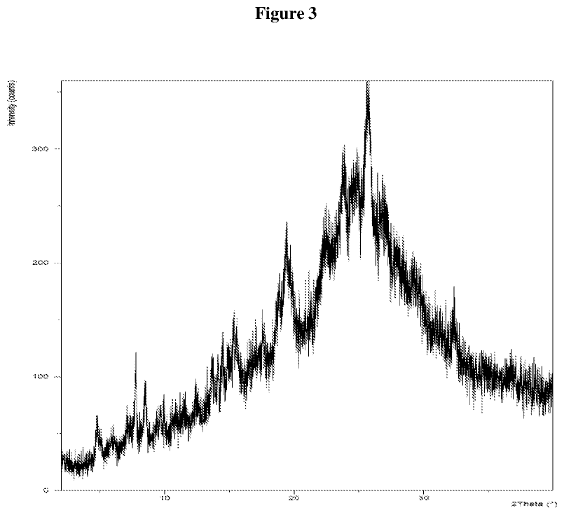

### DESCRIPTION OF THE EMBODIMENTS

The term “solution” as used herein means an appropriate mixture for purposes disclosed herein of one or more solutes in one or more solvents. Solution encompasses homogeneous mixtures as well as heterogeneous mixtures, such as slurries or other suspension mixtures having insoluble (not dissolved) material.

The present invention provides a novel compound of formula (IV).

In another aspect, the present invention provides use of compound of formula (IV) for the preparation of compound of formula I.

The present invention further provides a novel compound of formula (IV) characterized in that the x-ray diffraction of the compound exhibits peak maxima of 2 theta angle at 6.0,10.7,12.0,14.9,17.5,18.7, 20.1,22.8,22.9,24.6,25.0, 25.0, 25.5, 27.3, 27.4±0.2.

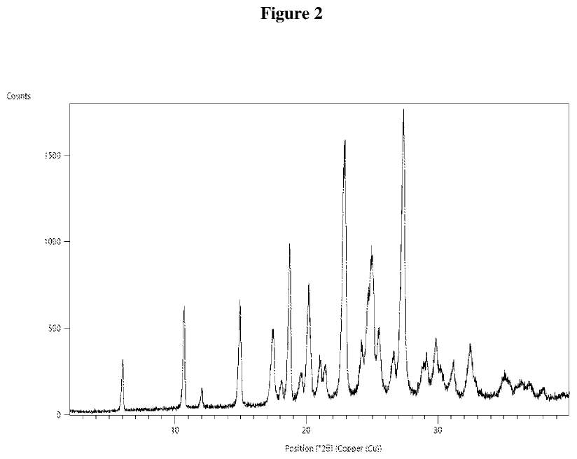

The present invention further provides a compound of formula (IV) as shown by the x-ray diffraction pattern of FIG. 2.

The present invention provides a novel process for preparing a compound of formula (IV), comprising reacting a compound of formula (III) in presence of ammonium formate in an alcohol and base to form a compound of formula (IV).

Ammonium formate may be used in an amount of 1 to 10 moles equivalent, suitably 1 to 3 moles equivalent, more preferably lor 2 or 3 moles equivalent. The reaction temperature may be usually 0° C. to 40° C., preferably 30° C. and the reaction temperature may be further raised to the reflux temperature of the solvent used. It may be suitable to conduct stirring for 1 to 10 hours, more suitably about 1 or 3 hours.

The alcohol solvent is selected form methanol, ethanol, n-propanol, isopropanol, n-butanol or tert-butanol and mixtures thereof, preferably methanol.

The base may be selected from sodium methoxide or sodium ethoxide, preferably sodium methoxide.

After cooling, the compound of formula (IV) (wet) can be isolated by using techniques such as, evaporation and/or filtration with or without gravity or suction, from the mass to obtain the desired product, and optionally the solid (wet) can be washed with a solvent. The compound of formula (IV) can be isolated by evaporating solvent and/or filtration. The solvent used for evaporation and/or filtration or washing may be selected from methanol, ethanol, n-propanol, isopropanol, butanol, ethyl acetate, methylenechloride, ethylenedichloride, water or mixture thereof.

The compound of formula (IV) after isolation can be dried at suitable temperature, at atmospheric or reduced pressure, for about 1-50 hours, or longer, using drying equipment, such as a tray dryer or vacuum oven. Drying temperature and time will be sufficient to achieve desired product. The temperature may be 30° C. to 70° C., preferably 45° C. to 55° C. for about 7 to 12 hours.

The present invention provides a novel process for the preparation of vericiguat compound of formula I comprising converting a compound of formula IV to vericiguat compound of formula I.

The scheme I illustrates individual reaction steps of present invention and illustrated by the way of examples.

Vericiguat or solvate thereof for the preparation of modification form II, III, monohydrate and dihydrate can be synthesized by the process of the present invention or may be obtained by any of the methods known in the literature.

The present invention provides a process for preparation of vericiguat modification form II comprising;
a) providing a solution of vericiguat or solvate thereof in a solvent;
b) adding a solvent;
c) isolating vericiguat modification Form II.

In one embodiment, vericiguat solvate may be selected from Vericiguat dimethyl sulfoxide, Vericiguat di-dimethyl sulfoxide and dimethylacetamide (DMAc), preferably vericiguat di-dimethyl sulfoxide solvate.

Vericiguat or solvate thereof, preferably vericiguat di-dimethyl sulfoxide solvate of step (a) may be dissolved in a solvent to form a solution.

The solvent for step (a) is selected from N-methyl pyrrolidone, N,N-dimethylformamide (DMF), dimethylsulfoxide (DMSO), dimethylacetamide (DMAc), preferably dimethylacetamide.

Vericiguat or solvate thereof, preferably vericiguat di-dimethyl sulfoxide may be dissolved or obtained in the solvent at a temperature of about 5° C. to the reflux temperature of the solvent. In another embodiment, Vericiguat or solvate thereof may be dissolved or obtained in the solvent, preferably dimethylacetamide, at 25 to 35° C. and the temperature may be further raised at 55 to 65° C. or 70 to 80° C.

After cooling below 5° C., preferably at 3° C., the solvent (antisolvent for precipitation) of step (b) may be selected form methyl acetate, ethyl acetate, chloroform, methylenechloride, ethylenedichloride, or mixture thereof, preferably ethyl acetate or methylenechloride.

After step b), the reaction mass of step (b) can be further isolated by filtration with or without gravity or suction, the obtained wet reaction mass can be washed with a solvent of step (b) and further suck dried to obtain vericiguat wet solid.

After washing, Vericiguat modification Form II can be isolated by drying vericiguat wet solid at suitable temperature ranges from 30° C. to 70° C., preferably 45° C. to 55° C., more preferably 50° C. The drying is perform using vacuum oven (Vacuum Tray dryer).

The present invention provides a process for preparation of vericiguat modification form II comprising;
I) providing a wet vericiguat or solvate thereof;
II) isolating vericiguat modification Form II.

Vericiguat or solvate thereof of step I) may be wet or dry solid. Vericiguat or solvate thereof may be selected form wet vericiguat solid. Wet Vericiguat solid comprises dimethylacetamide (DMAc) and ethyl acetate.

Vericiguat modification Form II can be isolated by drying wet vericiguat or solvate thereof at suitable temperature ranges from 50° C. to 90° C., preferably 75° C., for 15 to 24 hours, preferably 18 hours. The drying is performed using fluidized bed dryer or vacuum oven (Vacuum Tray dryer).

In further embodiment, the process for preparation of a wet vericiguat or solvate thereof, as used in above aspect of step (I) comprising;
i) providing a solution of vericiguat or solvate thereof in a solvent;
ii) combining a solvent to form wet solid;
iii) providing a wet solid from the wet solid of step ii in solvent.
iv) providing a wet vericiguat or solvate thereof from the wet solid of step iii in a solvent.

The solvate may be vericiguat dimethyl sulfoxide, Vericiguat di-dimethyl sulfoxide solvate.

The vericiguat or solvate thereof of step (i) may be dissolved in a solvent to form a solution or suspension, preferably solution.

The solvent of step (i) is selected from N-methyl pyrrolidone, N,N-dimethylformamide (DMF), dimethylacetamide (DMAc); or mixtures thereof, preferably dimethylacetamide.

The Vericiguat or its solvate, preferably Vericiguat di-dimethyl sulfoxide solvate may be dissolved or obtained in the solvent, preferably dimethylacetamide at a temperature of about 5° C. to the reflux temperature of the solvent used, preferably 25 to 35° C. and temperature may be further raised at 70° C. to 80° C., preferably 75° C., followed by filtration and washing with hot solvent, preferably dimethylacetamide (DMAc).

After filtration, reaction mass may be cooled below 5° C., preferably at 0° C. After cooling, the solvent of step (ii) is selected from chloroform, methylenechloride, ethylenedichloride, or mixture thereof, preferably methylenechloride.

After precipitation, the step (ii) further includes filtration of reaction mass of step (ii) and further dried to obtain wet solid. The wet solid comprises of vericiguat or solvate thereof, preferably wet solid of vericiguat, more preferably wet solid of vericiguat comprising methylene chloride and dimethylacetamide.

Step iii) provides a wet solid from the wet solid of step ii) in a solvent. The solvent for step iii) is selected from ester, preferably ethyl acetate.

The vericiguat wet solid of step ii) mixed in a solvent under stirring to form a suspension. Vericiguat wet solid of step ii mixed under stirring in a solvent i.e. ethyl acetate to form a suspension at a temperature of about 5° C. to the reflux temperature of the solvent, preferably at 30 to 35° C., more preferably 33° C. and stirred for 1 to 6 hours, preferably 2 to 3 hours.

The reaction mass of step iii) can be further isolated by using filtration with or without gravity or suction to obtain wet solid. The obtained wet solid can be washed with a solvent of step iii) and further suck dried to obtain wet solid. The wet solid comprises vericiguat or solvate thereof. The obtained wet vericiguat or its solvate comprising ethyl acetate and dimethylacetamide.

Step iv) provides a wet solid from the wet solid of step iii) in a solvent. The solvent for step iv) is selected from ester, preferably ethyl acetate.

The wet vericiguat or solvate thereof of step iii) is mixed under stirring in a solvent to form a suspension.

Vericiguat or solvate thereof i.e. vericiguat wet solid may be mixed under stirring in a solvent i.e. ethyl acetate to form a suspension at a temperature of about 5° C. to the reflux temperature of the solvent, preferably at 30 to 35° C., more preferably 33° C. and stirred for 1 to 6 hours, preferably 2 to 3 hours.

After formation of suspension, the reaction mass (suspension) can be further filtered with or without gravity or suction, the obtained wet solid can be washed with a solvent of step iv) and further suck dried to obtain wet solid. The wet solid comprises vericiguat or solvate thereof. The obtained wet vericiguat or its solvate comprising dimethylacetamide (DMAc) and ethyl acetate.

In one aspect, the present invention provides a process for preparation of vericiguat modification form III comprising;
a) providing a suspension of vericiguat or solvate thereof in a solvent
b) heating the solution
c) isolating vericiguat modification Form III.

The solvate may be Vericiguat dimethyl sulfoxide, Vericiguat di-dimethyl sulfoxide solvate, dimethylacetamide (DMAc). The solvate may be Vericiguat di-dimethyl sulfoxide solvate.

The vericiguat or solvate thereof of step (a) may be mixed in a solvent to form a solution or suspension, preferably suspension.

The solvent for step (a) is selected from alcohol. The alcohol may be selected form methanol, ethanol, n-propanol, isopropanol, butanol, or mixture thereof; preferably methanol.

In another embodiment, the Vericiguat or solvate thereof, preferably Vericiguat di-dimethyl sulfoxide is heated or obtained in the solvent, preferably methanol, at a temperature of about to the reflux temperature of the solvent used.

After cooling, the obtained wet solid containing vericiguat modification Form III can be isolated by filtration with or without gravity or suction, and further the solid can be washed with a solvent of step (a). After washing, Vericiguat modification Form III can be further isolated by drying using vacuum oven (Vacuum Tray dryer), at suitable temperature such as 30° C. to 70° C., preferably 45° C. to 55° C., preferably 50° C. for about 5 to 10 hours, preferably about 8 hours.

In one aspect, the present invention provides a process for preparation of vericiguat monohydrate comprising;
a) providing a solution of vericiguat or solvate thereof in a solvent
b) adding a mixture of solvent
c) isolating vericiguat monohydrate.

The vericiguat of step (a) may be dissolved in a solvent to form a solution or suspension, preferably solution.

In one embodiment, the solvent of step (a) includes acid such as formic acid.

The solution of step a) may be filtered through hyflow bed. The hyflobed may be washed with formic acid.

The solvent used in step (c) includes a mixture of liquor ammonia and water. The solvent of step (a) is added to step (b). The solvent of step (a) is added to step (b) at a temperature below 4° C., preferably at 2° C.

Vericiguat monohydrate of step (c) can be isolated by filtration with or without gravity or suction, and the solid can be washed with a solvent such as water. After washing, vericiguat monohydrate after isolation can be dried at suitable temperature, using a tray dryer, Drying temperatures will be sufficient to achieve desired product. The drying temperature may be at room temperature.

In one aspect, the present invention provides a process for preparation of vericiguat dihydrate comprising;
a) providing a solution of vericiguat or solvate thereof in a solvent
b) adding a solvent
c) isolating vericiguat dihydrate.

In one embodiment, the vericiguat or solvate thereof, preferably vericiguat may be mixed or obtained in the solvent at a temperature of about 20° C. to 35° C. temperature of the solvent used, and more preferably at about 27° C. to form a solution or suspension, preferably suspension.

In another embodiment, the solvent for step (a) is selected from methanol, ethanol, n-propanol, isopropanol, butanol or mixture thereof, preferably methanol.

The solvent used in step (b), can be added at about 27±3° C. The solvent used in step (b) include an aqueous ammonia.

Vericiguat dihydrate of step (c) can be isolated by using filtration with or without gravity or suction, and the solid can be washed with a solvent such as methanol.

After washing, Vericiguat dihydrate after isolation can be dried at suitable temperature, using vacuum oven (Vacuum Tray dryer). Drying temperature will be sufficient to achieve desired product. The temperature may be at 30 to 70° C., preferably 45 to 55° C.

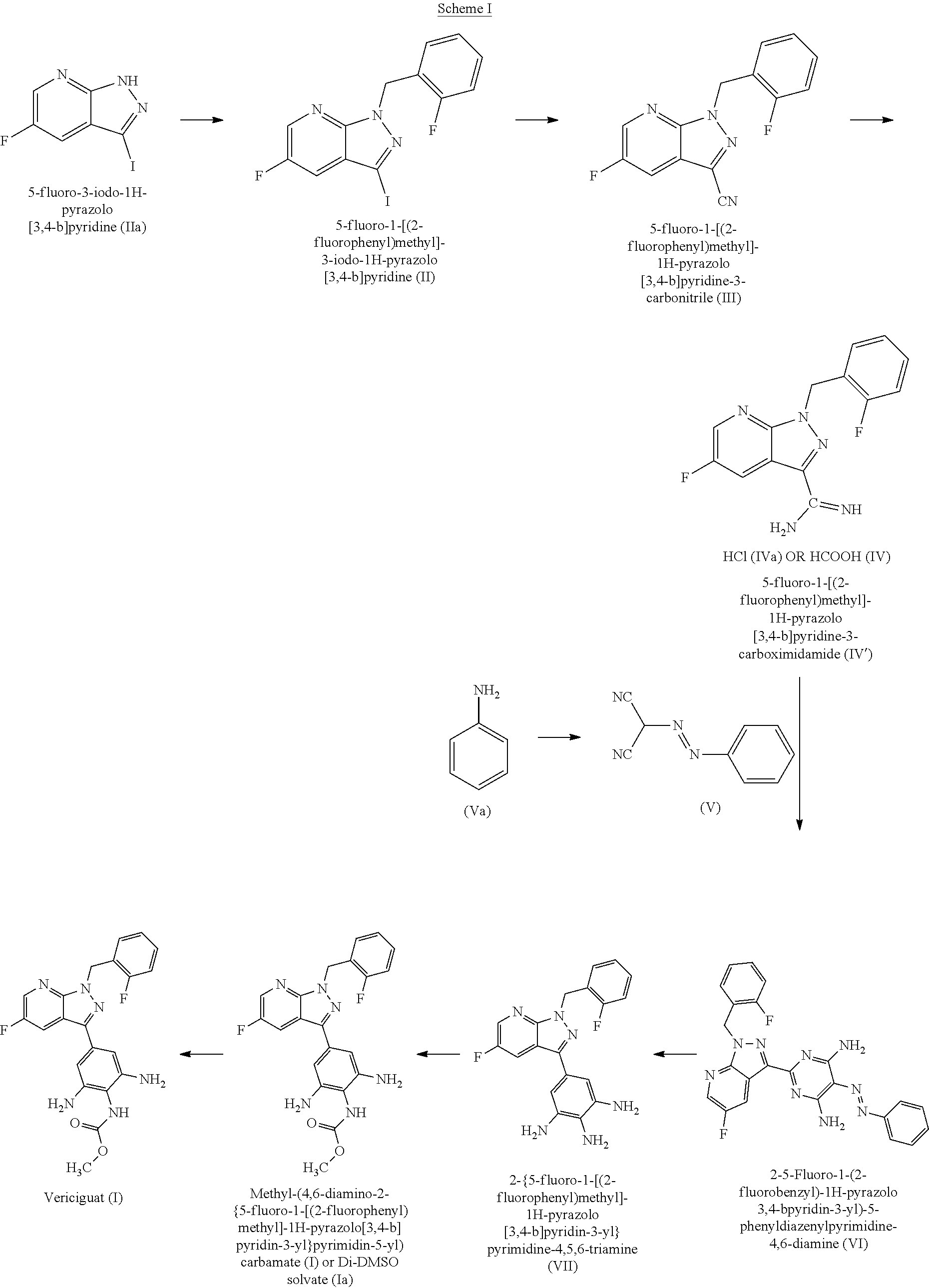

The present invention is further illustrated by the following examples which is provided merely to be exemplary of the invention and do not limit the scope of the invention. Certain modification and equivalents will be apparent to those skilled in the art and are intended to be included within the scope of the present invention.

### EXAMPLES

### Example 1: Preparation of 5-fluoro-1-[(2-fluorophenyl)methyl]-3-iodo-1H-pyrazolo[3,4-b]pyridine (II)

1000 ml of dimethylfumarate was charged into round bottom flask & charged 200.0 gm of 5-fluoro-3-iodo-1H-pyrazolo[3,4-b]pyridine. Reaction mass was stirred to get clear solution at 25° C. 235.37 gm of cesium carbonate was charged in reaction mass and stirred at 25° C. 136.55 gm of 2-fluoro benzyl bromide was charged and stirred the reaction mass at 25±5° C. and further stirred for 2 hrs at 25° C. 6000.0 ml of water was charged in the reaction mass maintaining temperature at 25±5° C. Reaction mass was stirred for 45-60 min at 25° C. Reaction mass was filtered and 1000 ml of water wash was given to wet solid. The solid was suck dried well and unload the wet cake. The wet solid was charged in 3200 ml of isopropyl alcohol and raise reaction temperature at 83° C. Reaction was stirred and maintained at 83° C. for 30-45 min. Heating was stopped and cooled the reaction mass at 25° C. Reaction mass was stirred at 25° C. for 45-60 min. Reaction mass was filtered and suck dried well. Unload the wet solid, wet wt. 218 gm. Wet product was dried in vacuum tray dryer at 50° C. for 8.0 hours. Dry weight of 5-fluoro-1-[(2-fluorophenyl)methyl]-3-iodo-1H-pyrazolo[3,4-b]pyridine-210 gm.

### Example 2: Preparation of 5-fluoro-1-[(2-fluorophenyl)methyl]-1H-pyrazolo[3,4-b]pyridine-3-carbonitrile (III)

1500.0 ml of dimethyl fumarate was charged into round bottom flask & charged 200.0 gm of 5-Fluoro-1 (2-fluorobenzyl)-3-iodo-1H-pyrazolo[3,4-b]Pyridine. Reaction mass was stirred at 30° C. 62.74 gm of copper cyanide was charged in reaction mass and reaction temperature was raised at 152° C. Reaction mass was stirred at 152° C. for 3.0 hrs. Heating was stopped and cooled the reaction mass at 30° C. Reaction mass was charged into solution of 1140.0 ml of aqueous ammonia and 6800.0 mL of water at 25° C. Reaction mass was stirred for 10-15 min at 30° C. Reaction mass was extracted with 2000 ml of ethyl acetate. Organic layer was separated and aqueous layer was again extracted with 2000 ml of ethyl acetate. Organic layer was separated and aqueous layer was discarded. Organic layer was filtered on hyflobed and the bed was washed with organic layer, followed by washing with 1000 ml of ethyl acetate. Combined filtrate and washed with 1000 ml of 2% Ethylene diamine tetra acetic acid solution at 30° C., followed by washing with 1000 ml of 10% sodium chloride solution. The organic layer was dried on 60.0 gm of sodium sulphate. Sodium sulphate was removed by filtration and washed with 1000 ml of ethyl acetate. Ethyl acetate was distilled out completely under vacuum at 52° C. Residue was co-distilled with 400 ml of n-heptane under vacuum at 52±3° C. followed by co-distilled with 400 ml of heptane under vacuum at 52° C. 400 ml of heptane was charged in residue and stirred for 30-45 minute at 52° C. Heating was stopped and cooled the reaction mass at 20° C. Reaction mass was stirred for 30-45 minute at 20° C. Reaction mass was filtered and washed 200 ml of heptane wash was given to wet solid, suck dried well and unload the wet solid wet wt. −148.0 gm. Wet solid was dried under vacuum at 50±3° C. for 10 hours. Dry wt: 130.0 gm.

### Example 3: Preparation of 5-fluoro-1-[(2-fluorophenyl)methyl]-1H-pyrazolo [3,4-b]pyridine-3-carboximidamide Formate (IV)

1000.0 ml of methanol was charged in the round bottom flask. 10.0 gm of sodium methoxide was charged in reaction mass at 30° C. The reaction mass was stirred for 10-20 minute at 30° C. 50.0 gm of 5-fluoro-1-[(2-fluorophenyl)methyl]-1H-pyrazolo[3,4-b]pyridine-3-carbonitrile was charged in reaction mass. The reaction mass was stirred for 3.0 hours at 30° C. 35.02 gm of ammonium formate was added in reaction mass at 30° C. The reaction temperature was raised at 63° C., stirred and maintained reaction mass at 63° C. The reaction mass was cooled below 55° C. temperature. The reaction mass was distilled under vacuum below 55° C. After distillation, degassing the residue under vacuum below 55° C. for 20-30 minute. 100.0 ml of ethyl acetate was charged in residue and stirred it at 52° C. Ethyl acetate was distilled out completely under vacuum below 55° C. 100.0 ml of ethyl acetate was charged in residue and stirred it at 52° C. Ethyl acetate was distilled out completely under vacuum below 55° C. Degassed the residue under vacuum completely below 55° C. 500.0 ml of methylene chloride was charged in residue below 55° C. The reaction mass was stirred at reflux temperature at 42° C. After heating, the reaction was cooled at 27° C. The reaction mass was stirred for 30-45 minute at 27° C. The reaction mass was filtered and washed the wet cake with 100.0 ml of methylene chloride. Suck dried the wet cake well. 100.0 gm of wet solid was charged in 500.0 ml of water. The reaction mass was stirred for 30-45 minute at 30° C. The reaction mass was filtered and washed the wet solid with 100.0 ml of water. Suck dried the wet solid well. Unload the wet solid wet wt. −100.0 gm. wet solid was dried in air tray dryer for 8-10 hours at 50° C. Unload the dry solid Dry wt. −58.0 gm (96.83%).

### Example 4: Preparation of 5-fluoro-1-[(2-fluorophenyl)methyl]-1H-pyrazolo [3, 4-b]pyridine-3-carboximidamide Formate (IV)

1250.0 ml of methanol was charged in the round bottom flask. 55.0 gm of sodium methoxide was charged in reaction mass at 30° C. The reaction mass was stirred for 10-20 minute at 30° C. 250.0 gm of 5-fluoro-1-[(2-fluorophenyl)methyl]-1H-pyrazolo[3,4-b]pyridine-3-carbonitrile was charged in reaction mass. The reaction mass was stirred for 3.0 hours at 30° C. 175.0 gm of ammonium formate was added in reaction mass at 30° C. The reaction temperature was raised at 63° C., stirred and maintained reaction mass at 63° C. The reaction mass was cooled below 30° C. temperature. The reaction mass was stirred for 1.0 hour at 30° C. Filtered the reaction mass and slurry wash with 250.0 ml of chilled methanol. 320.0 gm of wet solid was charged in 1250.0 ml of water. The reaction mass was stirred for 1 hour at 30° C. The reaction mass was filtered and washed the wet solid with 250.0 ml of water. Suck dried the wet solid well. Unload the wet solid wet wt. 440.0 gm. wet solid dried in air tray dryer for 10-12 hours at 50° C. Unload the dry solid. Dry wt. −290.0 gm (96.83%).

### Example 5: Preparation of 5-fluoro-1-[(2-fluorophenyl)methyl]-1H-pyrazolo [3, 4-b]pyridine-3-carboximidamide Hydrochloride (IVa)

2400.0 ml of methanol was charged in the round bottom flask. 23.99 gm of sodium methoxide was charged in reaction mass at 30±3° C. The reaction mass was stirred for 10-20 minute at 30° C. 120.0 gm of 5-fluoro-1-[(2-fluorophenyl)methyl]-1H-pyrazolo[3, 4-b]pyridine-3-carbonitrile was charged in reaction mass. The reaction mass was stirred for 3.0 hours at 30° C. 28.51 gm of ammonium chloride was charged in reaction mass at 30° C. The reaction temperature was raised at 63±3° C., stirred and maintained reaction mass at 63° C. and cooled the reaction mass below 55° C. temperature. The reaction mass was distilled under vacuum below 55° C. After distillation, degassed the residue under vacuum below 55° C. for 20-30 min. 240.0 ml of Ethyl acetate was added in residue and stirred it at 52° C. Ethyl acetate was distilled out completely under vacuum below 55° C. 240.0 ml of Ethyl acetate was charged in residue and stirred it at 52° C. Ethyl acetate was distilled out completely under vacuum below 55° C. Degassed the residue under vacuum completely below 55° C. 1200 ml of methylene chloride was charged in residue below 55° C. The reaction mass was stirred at reflux temperature at 42° C. The reaction was cooled at 27° C. The reaction mass was stirred for 30-45 minute at 27° C. The reaction mass was filtered and washed the wet cake with 120.0 ml of methylene chloride. Suck dried the wet cake well. Unloaded the wet cake wet wt.:−154.0 gm. 154.0 gm of wet solid was charged with 1200.0 ml of water. The reaction mass was stirred for 30-45 min at 30° C. The reaction mass was filtered and the wet solid was washed with 240 ml of water. Suck dried the wet solid well. Unloaded the wet solid wet wt.:−145.0 gm. Wet solid was dried in air tray dryer for 8-10 hours at 50° C. Unloaded the dry solid. Dry wt.:−103.0 gm (71.65%).

### Example 6: Preparation of 5-fluoro-1-[(2-fluorophenyl)methyl]-1H-pyrazolo [3, 4-b]pyridine-3-carboximidamide Hydrochloride (IVa)

2400.0 ml of methanol was added in the round bottom flask. 23.99 gm of sodium methoxide was added in reaction mass at 30° C. The reaction mass was added for 10-20 minute at 30° C. 120.0 gm of 5-fluoro-1-[(2-fluorophenyl)methyl]-1H-pyrazolo[3, 4-b]pyridine-3-carbonitrile was added in reaction mass. The reaction mass was stirred for 3.0 hours at 30° C. 71.27 gm of ammonium chloride was added in reaction mass at 30° C. and raised reaction temperature at 63° C., stirred and maintained reaction mass at 63° C. The reaction mass was cooled below 55° C. temperature. The reaction mass was distilled under vacuum below 55° C. After distillation, degassed the residue under vacuum below 55° C. for 20-30 minute. 240.0 ml of ethyl acetate was added in residue and stirred it at 52° C. Distilled out ethyl acetate completely under vacuum below 55° C. 240.0 ml of ethyl acetate was charged in residue and stir it at 52° C. Ethyl acetate was completely distilled out under vacuum below 55° C. Degassed the residue under vacuum completely below 55° C. 1200 ml of methylene chloride was charged in residue below 55° C. Stirred the reaction mass at reflux temperature i.e. 42° C. The reaction was cooled at 27° C. The reaction mass was stirred for 30-45 minute at 27° C. The reaction mass was filtered and washed the wet cake with 240.0 ml of methylene chloride. Suck dried the wet cake well. Unloaded the wet cake wet wt.:−232.0 gm. 232.0 gm of wet solid was charged with 1200.0 ml of water. The reaction mass was stirred for 30-45 minute at 30° C. The reaction mass was filtered and washed the wet solid with 240 ml of water. Suck dried the wet solid well. Unloaded the wet solid wet wt.:−170.0 gm. Wet solid was dried in air tray dryer for 8-10 hours at 50° C. Unloaded the dry solid. Dry wt.:−116.0 gm (80.69%).

### Example 7: Preparation of 2-5-Fluoro-1-(2-fluorobenzyl)-1H-pyrazolo[3, 4-b] pyridin-3-yl)-5-1 (E)-phenyldiazenylpyrimidine-4, 6-diamine (VI)

### a) Preparation of [(E)-phenyldiazenyl]malanonitrile] (V)

28.26 ml aniline was charged with 864 ml of water stirred it at 30±5° C. The reaction mass was cooled at 0-5° C. The aqueous solution of hydrochloric acid (56.18 ml of Conc. HCl diluted with 60.0 ml of water) was added slowly in reaction mass. The reaction mass was stirred for 5-15 minute at 0-5° C. Sodium nitrite solution was added (21.36 gm of sodium nitrite dissolve in 159.0 ml of water) slowly by addition funnel to control temperature at 0-5° C. The reaction mass was stirred for 5-15 min. at 0-5° C. Sodium acetate solution (32.23 gm of sodium acetate dissolve in 159.0 ml of water) was added slowly by addition funnel to control temperature at 0-5° C. The reaction mass was stirred for 10-20 min. at 0-5° C. Malononitrile solution (20.45 gm of malononitrile dissolve in 90.0 ml of methanol) was slowly added by addition funnel to control temperature at 0-5° C. The reaction mass was stirred for 1.50±0.30 min. at 0-5° C. The reaction mass was filtered and suck dried well. The wet cake was washed with 150.0 ml of chilled water. The wet cake was washed with 100.0 ml of chilled toluene. Suck dried the wet solid well and unloaded the wet solid. Unloaded the wet solid wet wt. −70.0 gm [(E)-phenyldiazenyl] malononitrile (Yellowish colour product).

### b) Preparation of [(E)-phenyldiazenyl]malanonitrile](V)

320.20 ml aniline was charged with 5040 ml of water stirred it at 30±5° C. The reaction mass was cooled at 0-5° C. The aqueous solution of hydrochloric acid (618.40 ml of Conc. HCl diluted with 700.0 ml of water) was added slowly in reaction mass. The reaction mass was stirred for 30 minute at 0-5° C. Sodium nitrite solution was added (242.1 gm of sodium nitrite dissolve in 1852 ml of water) slowly by addition funnel to control temperature at 0-5° C. The reaction mass was stirred for 30 minute at 0-5° C. Sodium acetate solution (363.6 gm of sodium acetate dissolve in 1852.0 ml of water) was added slowly by addition funnel to control temperature at 0-5° C. The reaction mass was stirred for 30 min. at 0-5° C. Malononitrile solution (231.7 gm of malononitrile dissolve in 1050.0 ml of Methanol) was slowly added by addition funnel to control temperature at 0-5° C. . . . The reaction mass was stirred for 2 hours at 0-5° C. The reaction mass was filtered and suck dried well. The wet cake was washed with 700.0 ml of chilled water. 1075.0 gm of wet solid was charged in 3500.0 ml of water. The reaction mass was stirred for 1 hour at below 30° C. The reaction mass was filtered and washed the wet solid with 1400.0 ml of water. Suck dried the wet solid well. 1038.0 gm of wet solid was charged in 1800.0 ml of toluene. The reaction temperature was raised at 45° C. The reaction mass was stirred for 1 hour at 45° C. The reaction mass was cooled below 30° C. temperature. The reaction mass was filtered and washed the wet solid with 400.0 ml of toluene. Suck dried the wet solid well. Unload the wet solid, wet wt. 800.0 gm. wet solid dried in vacuum tray dryer for 8-12 hours at 40° C. Unload the dry solid, Dry wt. −512.0 gm.

### c) Condensation of 5-fluoro-1-[(2-fluorophenyl)methyl]-1H-pyrazolo[3,4-b]pyridine-3-carboximidamide hydrochloride (IVa) and [(E)-phenyldiazenyl]malononitrile] (V)

120.0 gm of 5-fluoro-1-[(2-fluorophenyl)methyl]-1H-pyrazolo[3,4-b]pyridine-3-carboximidamide hydrochloride was charged with 600.0 ml of dimethylformamide and stirred it at 30° C. The reaction mass temperature was raised at 87° C. 77.50 ml of triethyl amine was slowly added in reaction mass at 87° C. [(E)-phenyldiazenyl]malononitrile (190.0 gm) was dissolve in 600.0 ml of dimethylfumarate (Weight of solution 660.0 gm) at 27° C. [(E)-phenyldiazenyl]malononitrile solution was slowly added in reaction mass at 87° C. After addition raised reaction mass temperature at 100° C. Reaction mass was stirred and maintained for 4-5 hours at 100° C. Heating was stopped and cooled the reaction mass at 27° C. 3600.0 ml of cool water was slowly added in reaction mass to control temp at 25° C. The reaction mass was cooled at 3° C. Reaction mass was stirred and maintained at 3° C. for 45-60 minute. Reaction mass was filtered and washed to wet solid with 600.0 ml of chilled water. Suck dried well. 120.0 ml of chilled methanol wash was given to wet solid and suck dried well. Unloaded the wet solid wet wt.:−180 gm. wet solid was dried in air tray dryer at 50° C. for 6.0 hours. Unloaded the solid Dry wt.:−132.0 gm (77.85%).

### d) Condensation of 5-fluoro-1-[(2-fluorophenyl)methyl]-1H-pyrazolo[3, 4-b]pyridine-3-carboximidamide formate (IV) with [(E)-phenyldiazenyl]malanonitrile] (V)

60.0 gm of 5-fluoro-1-[(2-fluorophenyl)methyl]-1H-pyrazolo[3,4-b]pyridine-3-carboximidamide formate was charged with 300.0 ml of dimethylformamide and stirred it at 30° C. The reaction mass temperature was raised at 87° C. 38.75 ml of triethyl amine was slowly added in reaction mass at 87° C. (E)-Phenyldiazenyl]malononitrile (80.0 gm) was dissolved in 300.0 ml of dimethylformamide at 27±3° C. (E)-Phenyldiazenyl] malononitrile solution was slowly added in reaction mass at 87° C. After addition, raised reaction mass temperature at 100° C. Reaction mass was stirred and maintained for 4-5 hours at 100° C. Heating was stopped and the reaction mass was cooled at 27° C. 1800.0 ml of cool water was slowly added in reaction mass to control temp at 25° C. The reaction mass was cooled at 3° C. Reaction mass was stirred and maintained at 3° C. for 45-60 minute. Reaction mass was filtered and washed to wet solid with 600.0 ml of chilled water. Suck dried well. 60.0 ml of chilled methanol wash was given to wet solid and suck dried well. Unloaded the wet solid wet wt. −89.0 gm. Wet solid was dried in air tray dryer at 50° C. The solid was dried at 50° C. till to get constant weight. A constant weight was obtained after 6.0 hours drying at 50° C. and heating was stopped. Unload the solid Dry wt. −78.0 gm (92%).

### e) Condensation of 5-fluoro-1-[(2-fluorophenyl)methyl]-1H-pyrazolo[3,4-b]pyridine-3-carboximidamide formate (IV) and [(E)-phenyldiazenyl]malononitrile] (V)

2000.0 ml of Toluene was charged in the round bottom flask. 150.0 gm of 5-fluoro-1-(2-fluorpbenzyl)-1H-pyrazolo (3,4-b) pyridine-3-carboximidamidamide formate was charged in reaction mass. 136.81 gm of potassium carbonate was charged in reaction mass. The reaction mass was stirred for 10-15 minute at 30° C. 91.90 gm of (E)-phenyldiazenyl]malanonitrile was charged in reaction mass. The reaction temperature was raised at 55° C., stirred and maintained reaction mass at 55° C. for 4-6 hours. The reaction mass was cooled below 35° C. temperature. 1500 ml of water was charged in reaction mass. The reaction mass was stirred for 1.0 hour at 30-35° C. Filtered the reaction mass and slurry washed with 450.0 ml of methanol to get wet solid. 280.0 gm of wet solid was charged in 1200.0 ml of Dimethylformamide. The reaction temperature was raised at 115° C., stirred and maintained reaction mass at 115° C. for 1 hour. The reaction mass was cooled below 35° C. temperature. The reaction mass was stirred for 1 hour at below 35° C. The reaction mass was filtered and washed the wet solid with 150.0 ml of methanol. 230.0 gm of wet solid was charged in 750.0 ml of water. The reaction mass was stirred for 1 hour at below 35° C. The reaction mass was filtered and washed the wet solid with 150.0 ml of methanol and 300 ml of water. Suck dried the wet solid well. Unload the wet solid wet wt. 310.0 gm. 310.0 gm of wet solid was charged in 750.0 ml of water. The reaction mass was stirred for 1 hour at below 35° C. The reaction mass was filtered and washed the wet solid with 150.0 ml of methanol and 300 ml of water. Suck dried the wet solid well. Unload the wet solid wet wt. 262.0 gm. wet solid dried in air tray dryer for 8-12 hours at 50° C. Unload the dry solid Dry wt. −190.0 gm (92.32%).

### Example 8: Preparation of 2-{5-fluoro-1-[(2-fluorophenyl)methyl]-1H-pyrazolo[3,4-b]pyridin-3-yl} pyrimidine-4,5,6-triamine (VII)

78.0 gm of 2-5-fluoro-1-(2-fluorobenzyl)-1H-pyrazolo[3,4-b]pyridin-3-yl)-5-1 (E)-phenyldiazenylpyrimidine-4,6-diamine was charged with 780.0 ml of Dimethylformamide in 2.0 L pressure vessel. 7.8 gm of palladium carbon catalyst was charged in reaction. The reaction mass was hydrogenated at 60-65° C. under 28-35 Kg/Cm2 Hydrogen atom for 8-10 hour. Heating was stopped and reaction mass was cooled to room temperature. The reaction mass was hold with nitrogen gas at 30° C. in reactor for overnight. Unloaded the reaction mass from reactor and reactor was washed with 70.0 ml of Dimethylformamide and washing was added to the reaction mass. Reaction mass was filtered through hyflow bed (Hyflow bed was prepared with 15.0 gm Hyflow powder in 100 ml of ethyl acetate). Hyflow bed was washed with 70.0 ml of Dimethylformamide and suck dried well. Filtrate was taken in 5.0 Litre round bottom flask and cooled to 22° C. 2730.0 ml of water was slowly added in reaction mass (clear solution) at 22±3° C. Reaction mass was stirred and maintained for 45-60 min at 22±3° C. Reaction mass was filtered and washed with 390.0 ml of water. Filtered solid was suck dried well and unloaded the wet solid wet wt. −72.0 gram. Arranged 2.0 Litre of 4.0 neck round bottom flask equipped with stirrer, thermopocket, condenser in a water bath. 720.0 ml of toluene was charged in 2.0 litre of 4 neck round bottom flask. 72.0 gm of crude wet solid was charged and stirred. The reaction temperature was raised at 55° C. Reaction mass was stirred and maintained at 55° C. for 45-60 minute. Heating was stopped and cooled the reaction mass at 27±3° C. Reaction mass was stirred and maintained for 45 to 60 minute at 27° C. Reaction mass was filtered and washed with 144.0 ml of toluene and suck dried well for 25-30 minute. Unloaded the solid wet wt. −70.0 gram. Wet solid was dried in vacuum tray dryer for 8.0 hours at 50° C. Unload the solid Dry wt. −50.0 Gm (80.64%).

### Example 9: Preparation of 2-{5-fluoro-1-[(2-fluorophenyl)methyl]-1H-pyrazolo[3,4-b]pyridin-3-yl} pyrimidine-4,5,6-triamine (VII)

50.0 gm of 2-5-fluoro-1-(2-fluorobenzyl)-1H-pyrazolo[3,4-b]pyridin-3-yl)-5-1 (E)-phenyldiazenylpyrimidine-4,6-diamine was charged with 1000.0 ml of dimethylformamide in 2.0 L pressure vessel. 2.5 gm of wet palladium carbon catalyst was charged in reaction. The reaction mass was hydrogenated at 60-65° C. under 8-10 Kg/Cm2 hydrogen atom for 8-10 hrs. Heating was stopped and reaction mass was cooled to 50° C. The reaction mass was hold with nitrogen gas. Reaction mass was filtered through hyflow bed (Hyflow bed was prepared with 15.0 gm Hyflow powder in 50 ml of dimethylformamide). Hyflow bed was washed with 100.0 ml of hot dimethylformamide and suck dried well. Cleared filtrate was taken in 2.0 litre round bottom flask and distilled the reaction mass completely under vacuum at about 60-80° C. Charged 50 ml of dimethylformamide in reaction mass and reaction mass was stirred and maintained at 75° C. for 30 minute. Heating was stopped and cooled the reaction mass at 27° C. Reaction mass was stirred and maintained for 1 hour at 27° C. Reaction mass was filtered and washed with 25 ml of dimethylformamide and suck dried well. Unloaded the solid wet wt. −70.0 gm. Wet solid was dried in vacuum tray dryer for 10.0 hours at 65° C. Unload the solid, Dry wt. −45.0 gm. 250.0 ml of isopropyl: water (25 ml: 225 ml) was charged in 500 ml of 4 neck round bottom flask. 45.0 gm of dry solid was charged and raised the reaction mass temperature to 83±3° C. Stirred and maintained the reaction mass for 1.5 hour at 83±3° C. Heating was stopped and cooled the reaction mass at 27±3° C. Reaction mass was stirred and maintained for 45 to 60 minute at 27° C. Reaction mass was filtered and washed with 100.0 ml of water and suck dried well. Unloaded the solid wet wt. −50.0 gm. Wet solid was dried in vacuum tray dryer for 10-12 hours at 65° C. Unload the solid Dry wt. −37.0 gm (91.9%).

### Example 10: Preparation of Methyl (4, 6-diamino-2-{5-fluoro-1-[(2-fluorophenyl)methyl]-1H-pyrazolo[3,4-b]pyridin-3-yl}pyrimidin-5-yl) Carbamate Di-DMSO Solvate (I and Ia)

20.0 2-{5-fluoro-1-[(2-fluorophenyl)methyl]-1H-pyrazolo[3,4-b]pyridin-3-yl} pyrimidine-4,5,6-triamine was charged with 200.0 ml of isopropyl alcohol. The reaction mass was stirred at 30° C. The reaction temperature was raised to 40° C. 5.85 ml of methyl chloroformate was added in reaction mass by addition funnel to control temperature at 40° C. The reaction mass was stirred and maintained at 40° C. for 4 hours. The reaction mass temperature was raised to 50° C. 50.0 ml of methanol was charged in reaction and maintain at 50° C. 13.09 ml of triethyl amine was added in reaction by addition funnel to control temperature at 50° C. After addition of triethyl amine, stirred and maintained the reaction mass for 45-60 minute at 50° C. Stopped heating and cooled the reaction mass at 27° C. The reaction mass was stirred and maintain for 45-60 minute, at 27° C. The reaction mass was filtered and suck dried well. The wet cake was washed with 40.0 ml of methanol. Suck dried well and wet solid wet wt −45.0 gm. The wet solid was dried in vacuum tray dryer at 50° C. for 8-10 hours. 22.00 gm of above solid was charged with 40.0 ml of dimethylsulfoxide. The reaction mass was stirred at 30° C. for 5-10 minute. 68.0 ml of ethyl acetate was charged in reaction mass. The reaction mass was raised at temperature 85° C. 1.5 gm (5%) activated carbon was charged in reaction mass during heating of reaction mass. Stirred and maintained reaction mass for 20-30 minute at 85° C. The reaction mass was filtered through hyflow bed (12.0 gm Hyflow powder in 40.0 ml Ethyl acetate) and the bed was washed with 40 ml of hot ethyl acetate. The filtrate was added into preheated 240.0 ml of ethyl acetate at 47° C. Stirred and maintained reaction mass for 50-60 minute at 47° C. The reaction mass was cooled within 65 minute up to at 27° C. The reaction mass was cooled within 65 minute to 0-5° C. Stirred and maintained reaction mass for 45-60 minute at 0-5° C. The reaction mass was filtered and washed with 26.0 ml×2 of ethyl acetate and suck dried well. Unload the wet solid wt.=32.0 gm. The wet solid was dried in vacuum tray dryer at 50° C. for 8-10 hours. Unload the solid Dry wt −15.00 gm (47.5%).

### Example 11: Preparation of methyl (4,6-diamino-2-{5-fluoro-1-[(2-fluorophenyl)methyl]-1H-pyrazolo[3,4-b]pyridin-3-yl}pyrimidin-5-yl)carbamate Di-DMSO solvate (I and Ia)

80.0 2-{5-fluoro-1-[(2-fluorophenyl)methyl]-1H-pyrazolo[3,4-b]pyridin-3-yl} pyrimidine-4,5,6-triamine was charged with 800.0 ml of isopropyl alcohol. The reaction mass was stirred at 30° C. The reaction temperature was raised to 40° C. 23.55 ml of methyl chloroformate was added in reaction mass by addition funnel to control temperature at 40° C. Raised the reaction temperature at 65° C. The reaction mass was stirred and maintained at 63±3° C. for 12 hours. 200.0 ml of methanol was charged in reaction and maintain at 65±3° C. for 1 hour. Stirred and maintained the reaction mass for 45-60 minute at 65±3° C. Stopped heating and cooled the reaction mass at 27° C. The reaction mass was stirred and maintain for 45-60 minute at 27° C. The reaction mass was filtered and washed with 160 ml of isopropyl alcohol. Suck dried well, wet wt. 150.0 gm. The wet solid was dried in vacuum tray dryer at 75° C. for 8-10 hours, dry wt. 96.0 gm. 163.2 ml of dimethylsulphoxide and 96.0 gm dry solid was charged in round bottom flask. The reaction temperature was raised at 75° C. 44.91 ml of diisopropyl ethylamine was added in reaction by addition funnel at 75° C. After addition of diisopropyl ethylamine, stirred and maintained the reaction mass for 30 minute at 75° C. 4.8 gm activated carbon was charged in reaction mass during heating of reaction mass. Stirred and maintained reaction mass for 30 minute at 75° C. The reaction mass was filtered through hyflow bed (38.4 gm Hyflow powder in 100.0 ml dimethylsulphoxide) and the bed was washed with 28.8 ml of hot dimethylsulphoxide. Charged the clear filtered reaction mass into round bottom flask. Cooled the reaction mass at 30° C. 672.0 ml of ethyl acetate was added in reaction mass at 30° C. Stirred and maintained reaction mass for 30 minute at 30° C. Raised the reaction mass temperature at 45° C. Stirred and maintained reaction mass for 45 minute at 45° C. The reaction mass was cooled at 30° C. Stirred and maintained reaction mass for 60 minute at 30° C. The reaction mass was cooled at 0° C. Stirred and maintained reaction mass for 60 minute at 0° C. The reaction mass was filtered and washed with 96.0 ml×2 of ethyl acetate and suck dried well. Unload the wet solid wt.=92.0 gm. The wet solid was dried in vacuum tray dryer at 75° C. for 8-10 hours. Unload the solid Dry wt −80.00 gm.

### Example 12: Preparation of Vericiguat Polymorph-I

320.0 ml of ethyl acetate was charged into the round bottom flask. 14.0 gm of methyl (4,6-diamino-2-{5-fluoro-1-[(2-fluorophenyl]-1H-pyrazolo[3,4-b]pyridine-3-yl}pyrimidin-5-yl) carbamate Di dimethylsulphoxide solvate was charged in reaction at 27° C. 77 ml of ethanol was added in reaction at 27° C. The reaction temperature was raised up to reflux at 72° C. Reaction mass was stirred and maintained at reflux 72° C. Heating was stopped after 16.0 hours, maintained and cooled the reaction at 27° C. The reaction mass was stirred at 27° C. for 25-30 minutes. Reaction mass was filtered and suck dried well. Wet cake was washed with 27 ml of ethyl acetate. Unloaded the wet solid wet wt. −14.70 gm. Wet solid was dried in vacuum tray dryer at 50° C. for 8-10 hours. Unload the dry solid Dry wt. −10.1 gm (98.56%).

### Example 13: Preparation of Polymorph Modification Form II of Vericiguat

15.0 gm Vericiguat didimethylsulphoxide solvate was charged in 75.0 ml of dimethylacetamide in the round bottom flask at 30° C. Reaction mass was stirred for 10.0 minutes at 30° C. Raised the reaction temperature to 60° C., stirred and maintained the reaction mass at 60° C. for 10 minute. The reaction mass was cooled to 3° C. 375.0 ml of ethyl acetate was added slowly to the reaction mass at 3° C. Reaction mass was stirred for 30 minutes. Reaction mass was filtered and washed with 75.0 ml of ethyl acetate and suck dried well. Unloaded the wet solid wt. −10.90 gm. Wet solid was dried in vacuum tray dryer for at 50° C. Unload the solid Dry wt −8.6 gm.

### Example 14: Preparation of Polymorph Modification Form II of Vericiguat

14.0 gm Vericiguat didimethylsulphoxide solvate was charged in 70.0 ml of dimethylacetamide in the round bottom flask at 27° C. Reaction mass was stirred for 5.0 minutes at 27° C. The reaction temperature was raised to 75° C., stirred and maintained the reaction mass at 75° C. for 15-20 minute. The reaction mass was slowly cooled to 3° C. Slowly added 350.0 ml of ethyl acetate to the reaction mass at 3° C. Reaction mass was stirred for 30-45 minutes. Reaction mass was filtered and washed with 70.0 ml of ethyl acetate and suck dried well. Unloaded the wet solid wt. −12.5 gm. Wet solid dried in vacuum tray dryer for at 50° C. Unload the solid Dry wt −8.0 gm.

### Example 15: Preparation of Polymorph Modification Form-II of Vericiguat

15.0 gm Vericiguat didimethylsulphoxide solvate was charged in 60.0 ml of dimethylacetamide in the round bottom flask at 30° C. Reaction mass was stirred for 10.0 minutes at 30° C. The reaction temperature was raised to 75° C., stirred and maintained the reaction mass at 75° C. for 10 minute and the reaction mass was cooled to 3° C. 375.0 ml of methylene dichloride was added to the reaction mass at 3° C. Reaction mass was stirred for 60 minutes. Reaction mass was filtered and washed with 75.0 ml of methylene dichloride and suck dried well. Unloaded the wet solid wt. −10.3 gm. Wet solid dried in vacuum tray dryer for at 50° C. Unload the solid Dry wt −7.3 gm.

### Example 16: Preparation of Polymorph Modification Form-II of Vericiguat

14.0 gm Vericiguat didimethylsulphoxide solvate was charged in 70.0 ml of dimethylacetamide in the round bottom flask at 27° C. Reaction mass was stirred for 5.0 minutes at 27° C. The reaction temperature was raised to 75° C., stirred and maintained the reaction mass at 75° C. for 15-20 minute. The reaction mass was slowly cooled to 3° C. 350.0 ml of methlenechloride was added to the reaction mass at 3° C. Reaction mass was stirred for 30-45 minutes. Reaction mass was filtered and washed with 70.0 ml of methlenechloride and suck dried well. Unloaded the wet solid wt. −12.5 gm. Wet solid was dried in vacuum tray dryer for at 50° C. Unload the solid Dry wt −8.5 gm.

### Example 17: Preparation of Polymorph Modification Form-II of Vericiguat

80.0 gm Vericiguat didimethylsulphoxide solvate was charged in 300.0 ml of dimethylacetamide in the round bottom flask at 30° C. Reaction mass was stirred for 10.0 minutes at 30° C. Raise the reaction temperature to 75° C., stirred and maintained the reaction mass at 75° C. for 30 minute. Fine filtered the reaction and wash with 20.0 ml hot dimethylacetamide. Charged clear filtrate in to round bottom flask. The reaction mass was cooled to 0° C. 2000.0 ml of methylene chloride was added slowly to the reaction mass at 0° C. Reaction mass was stirred for 1-2 hours at 0° C. Reaction mass was filtered and washed with 240.0 ml of methylene chloride. Suck dried well. wet solid wt. 180.0 gm. 180.0 gm wet solid and 1200.0 ml of ethyl acetate was charged in to round bottom flask. Reaction mass was stirred for 2-3 hours at 33° C. Reaction mass was filtered and washed with 160.0 ml of ethyl acetate. Suck dried well. Unloaded the wet solid wt. 164.0 gm. 164.0 gm wet solid and 1200.0 ml of ethyl acetate was charged in to round bottom flask. Reaction mass was stirred for 2-3 hours at 33° C. Reaction mass was filtered and washed with 160.0 ml of ethyl acetate. Suck dried well. Unloaded the wet solid wt. 150.0 gm Wet solid was dried in vacuum tray dryer for at 75° C. for 18 hours. Unload the solid, dry wt-85.0 gm.

### Example 18: Preparation of Polymorph Modification Form-III of Vericiguat

10.0 g of Vericiguat didimethylsulphoxide solvate mixed in 250.0 ml of methanol at 27° C. Heated to reflux and maintained under reflux for 14 hours. The reaction mass was cooled to 27° C. and filtered the solid. The solid was washed with 30 ml of methanol. Wet wt: 11.0 g. Dried the solid in vacuum tray dryer at 50° C. for 8 hours. Dry wt: 8.0 gm.

### Example 19: Preparation of Vericiguat Monohydrate

2.0 gm of Vericiguat was dissolved in 6.0 ml of formic acid. The solution was filtered through hyflow bed and the bed was washed with 2.0 ml of formic acid. Mixture of 25.0 ml of liquor ammonia and 60.0 ml of water was added in the formic acid solution at 2° C. and stirred at 2° C. for 10 minute. The solid was filtered and washed with 60 ml of water, dried in air tray dryer at room temperature. Dry wt: 2.20 gm.

### Example 20: Preparation of Vericiguat Dihydrate

2.0 gm of Vericiguat was dissolved in 16.0 ml of methanol at 27° C. 0.8 ml of aqueous ammonia solution was slowly added to the reaction mass at 27° C. Reaction mass was stirred for 3.0 hours at 27° C. The solution was filtered and washed with 4.0 ml of methanol and dried in vacuum tray dryer at 50° C. Dry wt: 1.10 gm.

### Example 21: Preparation of Vericiguat (I)

15.0 gm 2-{5-fluoro-1-[(2-fluorophenyl)methyl]-1H-pyrazolo[3,4-b]pyridin-3-yl}pyrimidine-4,5,6-triamine was charged in 285.0 ml of pyridine and stirred at 30° C. under N₂ atmosphere. The reaction mass was cooled at 0-5° C. under N₂ atmosphere. 3.15 ml of methylchloroformate in methylene dichloride was slowly added in reaction mass to control temperature at 0-5° C. Reaction mass was stirred and maintained at 0-5° C. under N₂ atmosphere. After 1.0 hours, maintaining at 0-5° C. the reaction mass temperature was raised to 25° C. and further maintained for 15 hours at 25° C. The reaction mass distilled out under reduced pressure. 50 ml toluene was added to reaction mass and distilled out at 40° C. 10 ml toluene was added and distilled out at 40° C. 10 ml toluene was again added and distilled out at 40° C. 20 ml ethanol/water 1:1 (20 ml) was added to residue at 30° C. The reaction mass was stirred for 30 minute at 30° C. The reaction mass was filtered at 30° C. and washed with ethanol:ethyl acetate mixture. Unload the solid (4.7 gm) and stirred in diethyl ether at 30° C. for 30 minute. The reaction mass was filtered and washed with diethyl ether. Unload the solid and dried under vacuum at 40° C. Dry wt: 14.9 gm.

1. 5-fluoro-1-[(2-fluorophenyl)methyl]-1H-pyrazolo [3, 4-b]pyridine-3-carboximidamide formate compound of formula (IV).

2. The compound of claim 1, wherein the x-ray diffraction of the compound exhibits 2 theta peak maxima of angle at 6.0,10.7,12.0,14.9,17.5,18.7,20.1,22.8,22.9,24.6,25.0, 25.0, 25.5, 27.3, 27.4±0.2.

3. Use of compound of formula (IV) for the preparation of vericiguat compound of formula I.

4. A process for preparing a compound formula (IV), comprising reacting a compound of formula (III) in presence of ammonium formate in an alcohol and base to form a compound of formula (IV).

5. The process according to claim 4, wherein the alcohol is selected from methanol, ethanol, n-propanol, isopropanol, n-butanol or tert-butanol and mixtures thereof.

6. The process according to claim 4 further comprises base selected form sodium methoxide, sodium ethoxide.

7. A process for preparing a vericiguat compound formula (I) comprises converting a compound of formula IV to vericiguat compound of formula I.

8. A process for preparation of vericiguat modification form II comprising;
a) providing a solution of vericiguat or solvate thereof in a solvent;
b) adding a solvent;
c) isolating vericiguat modification Form II.

9. The process according to claim 8, wherein the solvent of step (a) is selected from N-methyl pyrrolidone, N,N-dimethylformamide (DMF), dimethylsulfoxide (DMSO), dimethylacetamide (DMAc); or mixture thereof, preferably dimethylacetamide.

10. The process according to claim 8, wherein the solvent of step b) is selected from methyl acetate, ethyl acetate, chloroform, methylenechloride, ethylenedichloride, or mixture thereof, preferably ethyl acetate or methylenechloride.

11. The process according to claim 8, wherein vericiguat solvate of step a) is selected from Vericiguat dimethyl sulfoxide, Vericiguat di-dimethyl sulfoxide solvate and dimethylacetamide (DMAc), preferably vericiguat didimethylsulphoxide solvate.

12. A process for preparation of vericiguat modification form II comprising;
I) providing a wet vericiguat or solvate thereof;
II) isolating vericiguat modification Form II.

13. The process according to claim 12, wherein wet vericiguat or solvate thereof comprising dimethylacetamide (DMAc) and ethyl acetate.

14. The process according to claim 12, wherein the drying temperature at which vericiguat modification Form II is isolated in the range of from 50° C. to 90° C., preferably 75° C.

15. A process according to claim 12, wherein preparation of wet Vericiguat or solvate thereof of step (I) comprising;
i) providing a solution of vericiguat or solvate thereof in a solvent;
ii) combining a solvent to form wet solid;
iii) providing a wet solid from the wet solid of step ii in a solvent.
iv) providing a wet vericiguat or solvate thereof from the wet solid of step iii in a solvent.

16. The process according to claim 15, wherein the solvent of step i) is selected from N-methyl pyrrolidone, N,N-dimethylformamide (DMF), dimethylacetamide (DMAc); or mixtures thereof, preferably dimethylacetamide.

17. The process according to claim 15, wherein vericiguat solvate of step I) is selected from vericiguat dimethyl sulfoxide, Vericiguat di-dimethyl sulfoxide solvate.

18. The process according to claim 15, wherein solvent of step (ii) is selected form chloroform, methylenechloride, ethylenedichloride, or mixture thereof, preferably methylenechloride.

19. The process according to claim 15, wherein solvent of step iii) and iv) is selected form ester, wherein ester is selected form ethyl acetate.

20. A process for preparation of vericiguat modification form III comprising;
a) providing a suspension of vericiguat or solvate thereof in a solvent;
b) heating the solution;
c) isolating vericiguat modification Form III.

21. The process according to claim 20, wherein the solvent of step a) is selected from alcohol, wherein alcohol is selected from methanol, ethanol, n-propanol, isopropanol, butanol or mixture thereof, preferably methanol.

22. The process according to claim 20, wherein heating the solution of step b) to the reflux temperature of the solvent used.

23. The process according to claim 20, wherein vericiguat solvate of step a) is selected from vericiguat didimethylsulphoxide solvate.

24. The process according to claim 20, wherein a drying temperature at which vericiguat modification form III is isolated in the range of from 30° C. to 70° C., preferably 45 to 55° C., more preferably 50° C.

25. A process for preparation of vericiguat monohydrate comprising;
a) providing a solution of vericiguat or solvate thereof in a solvent;
b) adding mixture of solvent;
c) isolating vericiguat monohydrate.

26. The process according to claim 25, wherein the solvent of step (a) comprises formic acid.

27. The process according to claim 25, wherein the solvent of step (c) comprises a mixture of liquor ammonia and water.

28. The process according to claim 25, wherein the solvent of step (a) is added to (b) at below 4° C., preferably at 2° C.

29. The process according to claim 25, wherein the drying temperature at which vericiguat monohydrate is isolated at room temperature.

30. A process for preparation of vericiguat dihydrate comprising;
a) providing a solution of vericiguat or solvate thereof in a solvent
b) adding a solvent;
c) isolating vericiguat dihydrate.

31. The process according to claim 30, wherein the solvent of step (a) is selected from methanol, ethanol, n-propanol, isopropanol, butanol or mixture thereof, preferably methanol.

32. The according to claim 30, wherein the solvent of step (b) is selected from aqueous ammonia.

33. The process according to claim 30, wherein the solvents of step (b) is added at 27±3° C.

34. The process according to claim 30, wherein step a) provides suspension.

35. The process according to claim 30, wherein the drying temperature at which vericiguat dihydrate is isolated in the range of from 30° C. to 70° C., preferably 45 to 55° C.
